(19) United States
(12) Patent Application Publication
(10) Pub. No.: US 2022/0125620 A1
(43) Pub. Date: Apr. 28, 2022

(54) THE POUCH AKA THE DRIPPER

(71) Applicants: Larry Carrington, Sacramento, CA (US); Kerrie Griffin, Sacramento, CA (US)

(72) Inventors: Larry Carrington, Sacramento, CA (US); Kerrie Griffin, Sacramento, CA (US)

(21) Appl. No.: 17/081,165

(22) Filed: Oct. 27, 2020

(51) Int. Cl. A61F 5/453; A61F 5/44; A61F 5/441

(52) U.S. Cl. CPC A61F 5/453; A61F 5/441; A61F 5/4407; A61F 5/4401

(57) ABSTRACT

An article of manufacture for providing a small urine drop catching pouch comprises a small pouch having an opening and an inner liner for placing over a penis after urinating and a closure for securing the opening once the drop catching pouch has been used. The drop catching pouch is made from a material comprising sheep skin, silk, help, cotton, and bamboo. The closure comprises elastic, string, and VELCRO™. The inner liner comprises an absorbent layer for retaining urine and eliminating odor comprising sodium polyacrylate granules and baking soda.

### TECHNICAL FIELD

This application relates in general to an article of manufacture for providing personal protection devices, and more specifically, to an article of manufacture for providing a small urine drop catching pouch.

### BACKGROUND

When a man uses the restroom and just when he thinks he has finished he has a few drops of urine that leaves a wet spot on his pants. The problem may cause serious embarrassment when it occurs in a work or social situation.

Therefore, a need exists for a an article of manufacture for providing a small urine drop catching pouch. The present invention attempts to address the limitations and deficiencies of prior solutions according to the principles and example embodiments disclosed herein.

### SUMMARY

In accordance with the present invention, the above and other problems are solved by providing an article of manufacture for providing a small urine drop catching pouch according to the principles and example embodiments disclosed herein.

In one embodiment, the present invention is an article of manufacture for providing a small urine drop catching pouch. The small urine drop catching pouch is made of a small pouch having an opening and an inner liner for placing over a penis after urinating and a closure for securing the opening once the drop catching pouch has been used. The drop catching pouch is made from a material comprising sheep skin, silk, help, cotton, and bamboo. The closure comprises elastic, string, and VELCRO™. The inner liner comprises an absorbent layer for retaining urine and eliminating odor comprising sodium polyacrylate granules and baking soda.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment for an article of manufacture for providing a small urine drop catching pouch according to the present invention.

FIG. 2a illustrates another example embodiment of an article of manufacture for providing a small urine drop catching pouch according to the present invention.

FIG. 2b illustrates an example embodiment of an article of manufacture for providing a small urine drop catching pouch according to the present invention.

### DETAILED DESCRIPTION

This application relates in general a system and method for providing a personal protection device, and more specifically, to an article of manufacture for providing a small urine drop catching pouch according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

The terms “customer,” and “user” refer to an entity, e.g. a human, using the a small urine drop catching pouch associated with the invention. The term user herein refers to one or more users.

The term “invention” or “present invention” refers to the invention being applied for via the patent application with the title “The Pouch aka The Dripper.” Invention may be used interchangeably with face mask.

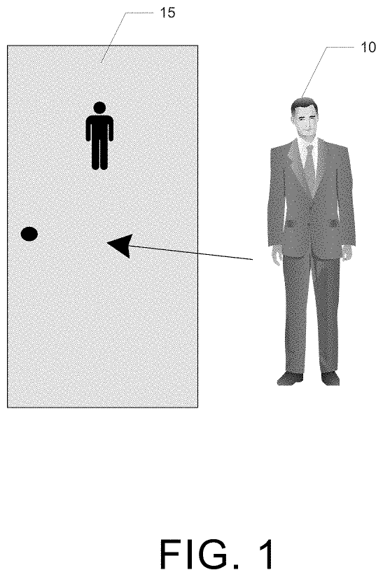

In general, the present disclosure relates a system and method for providing a personal protection device. To better understand the present invention, FIG. 1 illustrates an example environment when an article of manufacture for providing a small urine drop catching pouch according to the present invention is useful. A user 10, while at work or an important social event, may need to visit a restroom 15. Because of the occasion, the user 10 may be wearing expensive clothing as he attempts to present himself at his best. The user 10 desires to remain looking his best when he leaves the restroom 15 once he has completed his business therein.

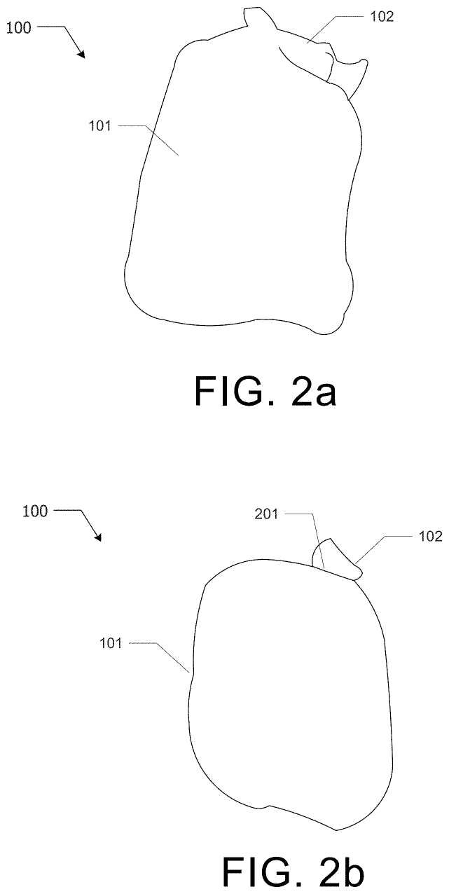

FIG. 2a illustrates an example embodiment of an article of manufacture for providing a small urine drop catching pouch according to the present invention. Before a man leaves the restroom 15, the present invention may be worn. A 2 inch pouch 101 that fits on the on the end of the penis to catch the extra drops of urine after he urinates. The pouch 101 has an opening at one end 102 having a one half to 1 inch closure. The pouch 101 is shaped like a condom with ridges around the rim that permits the user to put it onto his penis. The pouch 101 is made of sheep skin, silk, help, cotton, and bamboo to retain any residual urine that may be present or leave the user's penis as he walks out of the restroom 15. Other materials that may be used include a liner that provides and absorbent layer. The absorbent layer could be made of Sodium Polyacrylate Granules. Baking soda may also be used to handle the smell of urine. The pouch 101 can possibly be sealed with elastic, string, or Velcro™.

FIG. 2b illustrates an example embodiment of an article of manufacture for providing a small urine drop catching pouch according to the present invention. In this second embodiment, the pouch 101 has had the opening 102 secured using a closing or tying device 201. Once the user 10 has completed his need for the pouch 101, the user may wish to prevent any residual urine within the pouch 101 to escape when it is being disposed. The opening 102 may be secured by tying a string or thread 201 around the material of the pouch 101 about the opening 102. The tying device 201 may be sewn within a channel about the opening 102 or merely attached to the pouch 101 and wrapped about the opening 102 when it has been closed by gathering together the material of the pouch 101 and tying the thread into a knot. Once secured, the closed pouch 101 of FIG. 2b may be thrown into the garbage for disposal.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. An article of manufacture for providing a small urine drop catching pouch, the drop catching pouch comprises:
a small pouch having an opening and an inner liner for placing over a penis after urinating; and
a closure for securing the opening once the drop catching pouch has been used.

2. The drop catching pouch according to claim 1, wherein the drop catching pouch is made from a material comprising sheep skin, silk, help, cotton, and bamboo.

3. The drop catching pouch according to claim 1, wherein the closure comprises elastic, string, and VELCRO™.

4. The drop catching pouch according to claim 1, wherein the inner liner comprises an absorbent layer for retaining urine and eliminating odor.

5. The drop catching pouch according to claim 4, wherein the absorbent layer comprises sodium polyacrylate granules and baking soda.
